(19) United States
(12) Patent Application Publication
(10) Pub. No.: US 2020/0300799 A1
(43) Pub. Date: Sep. 24, 2020

(54) REDUCING GAS SENSOR

(71) Applicant: CANON KABUSHIKI KAISHA, Tokyo (JP)

(72) Inventors: Akira Tsuboyama, Machida-shi (JP); Koji Yano, Tokyo (JP)

(21) Appl. No.: 16/896,832

(22) Filed: Jun. 9, 2020

Related U.S. Application Data
(63) Continuation of application No. PCT/JP2018/045140, filed on Dec. 7, 2018.

(30) Foreign Application Priority Data
Dec. 15, 2017 (JP) 2017-240947

(51) Int. Cl. G01N 27/30; G01N 27/04; G01N 33/00; H01M 8/04313; C07F 15/00

(52) U.S. Cl. CPC G01N 27/30; G01N 27/04; C07F 15/0066; H01M 8/04313; G01N 33/005

(57) ABSTRACT

A pair of electrodes; and a reactive layer electrically in contact with the pair of electrodes are included. The reactive layer includes a palladium metal complex represented by General formula (1) below, and a change in electric conductivity between the pair of electrodes is measured to detect a reducing gas. The change is caused by an irreversible redox reaction between the palladium metal complex and the reducing gas.

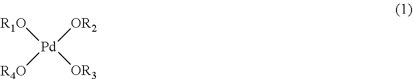

where each of OR₁ to OR₄ is a monodentate ligand, or forms a polydentate ligand by bonding to each other, or forms a bridging ligand by bonding to a palladium atom different from the palladium atom in General formula (1), and R₁ to R₄ each have one or more carbons and may be the same or different.

### CROSS-REFERENCE TO RELATED APPLICATIONS

This application is a Continuation of International Patent Application No. PCT/JP2018/045140, filed Dec. 7, 2018, which claims the benefit of Japanese Patent Application No. 2017-240947, filed Dec. 15, 2017, both of which are hereby incorporated by reference herein in their entirety.

### BACKGROUND OF THE INVENTION

### Field of the Invention

The present invention relates to a reducing gas sensor configured to detect a reducing gas.

### Description of the Related Art

The reducing gas is a compound that has a property of having high reducibility and reducing a compound susceptible to reduction upon contact with the compound, and that is gaseous at ordinary temperature. Specific examples of the reducing gas include hydrogen, formaldehyde, carbon monoxide, and ethylene. These reducing gases are industrially and widely used, but some of them are flammable, explosive, or affect the human body when inhaled. Thus, for safety control, leakage, to the outside, of reducing gases from storage, for example, tanks, cylinders, or pipes needs to be detected.

Japanese Patent Application Laid-Open No. 2013-540998 describes, as a sensor that detects hydrogen gas as a reducing gas, a color changing sensor that changes color upon reaction with hydrogen gas. Specifically, the sensor has, in its reaction section, a coloring material that changes its color upon reaction with hydrogen. As such coloring materials, platinum group metal compounds (such as platinum (Pt), palladium (Pd), and iridium (Ir)) are described.

Japanese Patent Application Laid-Open No. 2009-8476 describes a hydrogen gas sensor that includes a pair of electrodes and a carbon nanomaterial (CNT) bridging the pair of electrodes, and that measures the conductance or resistance between the electrodes. In Japanese Patent Application Laid-Open No. 2009-8476, the surface of the CNT is modified with a catalytic metal material. The catalytic metal material modifying the CNT includes catalytic metal particles precipitated by a redox reaction between the catalytic metal and the CNT immersed in a solution of a salt or complex of the catalytic metal, and catalytic metal particles precipitated by a redox reaction between the electrode metal and the catalytic metal. The CNT is modified with the catalytic metal material to cause an increase in the change in the Schottky barrier height between the CNT and the electrode metal upon contact with hydrogen gas, which results in an increase in the change in the conductance or resistance between the electrodes. Japanese Patent Application Laid-Open No. 2009-8476 describes, as the catalytic metal material, Pd acetate.

Nanotechnology, 21, 165503 (5 pp), 2010 describes a hydrogen gas sensor having, in its reaction section, Pd oxide. In Nanotechnology, 21, 165503 (5 pp), 2010, the sensor detects a change in the conductance of a Pd oxide film due to a reversible redox reaction with hydrogen gas.

In order to detect a reducing gas in the air, a sensor that detects the reducing gas at high sensitivity and accuracy is necessary.

However, the sensor of Japanese Patent Application Laid-Open No. 2013-540998 has uncertainty because the user of the coloring material visually judges the degree of the color change. There is a method of optically detect the color change, which may result in an increase in the size of the sensor. In addition, since the color change is employed, the sensor needs to be disposed in a place where the color change is observable by the user.

In such a reducing gas sensor in Japanese Patent Application Laid-Open No. 2009-8476 or Nanotechnology, 21, 165503 (5 pp), 2010 that uses changes in the electric characteristics of the reactive layer, the sensitivity is one of important parameters that dictate the power consumption of the sensor. When the sensor has high sensitivity, the conductance considerably changes before and after the reaction section comes into contact with the reducing gas, and the electric conductivity is low prior to the reaction with the reducing gas. This enables a reduction in the power consumption in the normal state.

In Japanese Patent Application Laid-Open No. 2009-8476, the rate of change in conductance calculated from normalized conductance (ΔG/G₀) before and after the reaction with hydrogen gas is about 10 to about 20%, which is not a sufficient sensitivity.

In Nanotechnology, 21, 165503 (5 pp), 2010, the sensitivity S of the hydrogen sensor is determined by the following Formula (1). The hydrogen sensor of Nanotechnology, 21, 165503 (5 pp), 2010 has a sensitivity S of about 45, which is not a sufficient sensitivity.

S=(G_{H}−G_{N})/G_{N} (1)

(G_{H}: conductance in the presence of hydrogen, G_{N}: conductance in the absence of hydrogen)

In consideration of such problems, an object is to achieve, in a sensor configured to detect a reducing gas, a higher sensitivity than in the related art, and a reduction in the power consumption.

### SUMMARY OF THE INVENTION

A sensor according to an aspect of the present invention includes a pair of electrodes and a reactive layer electrically in contact with the pair of electrodes, wherein the reactive layer includes a palladium metal complex represented by General formula (1) below, and the sensor is configured to measure a change in electric conductivity between the pair of electrodes, the change being caused by an irreversible redox reaction between the palladium metal complex and the reducing gas, to detect the reducing gas.

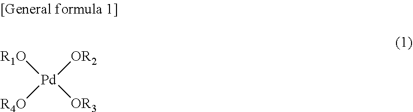

In General formula (1), each of OR₁ to OR₄ is a monodentate ligand, or forms a polydentate ligand by bonding to each other, or forms a bridging ligand by bonding to a palladium atom different from the palladium atom in General formula (1). R₁ to R₄ each have one or more carbons, and may be the same or different.

Further features of the present invention will become apparent from the following description of exemplary embodiments with reference to the attached drawings.

### BRIEF DESCRIPTION OF THE DRAWINGS

FIG. 1A is a schematic view illustrating the configuration of a sensor according to a first embodiment.

FIG. 1B is a schematic view illustrating the configuration of a sensor according to a first embodiment.

FIG. 2 is a schematic view illustrating an example of the molecular structure of a palladium metal complex according to a first embodiment.

FIG. 3 is a schematic view illustrating the configuration of a sensor according to a second embodiment.

FIG. 4 is a schematic view illustrating the configuration of a fuel cell car according to a third embodiment.

FIG. 5 is a block diagram illustrating the configuration of a hydrogen gas station according to a third embodiment.

### DESCRIPTION OF THE EMBODIMENTS

Hereinafter, embodiments and Examples according to the present invention will be described. Incidentally, the present invention is not limited to the following embodiments and Examples, which can be modified within the scope of the invention.

### First Embodiment

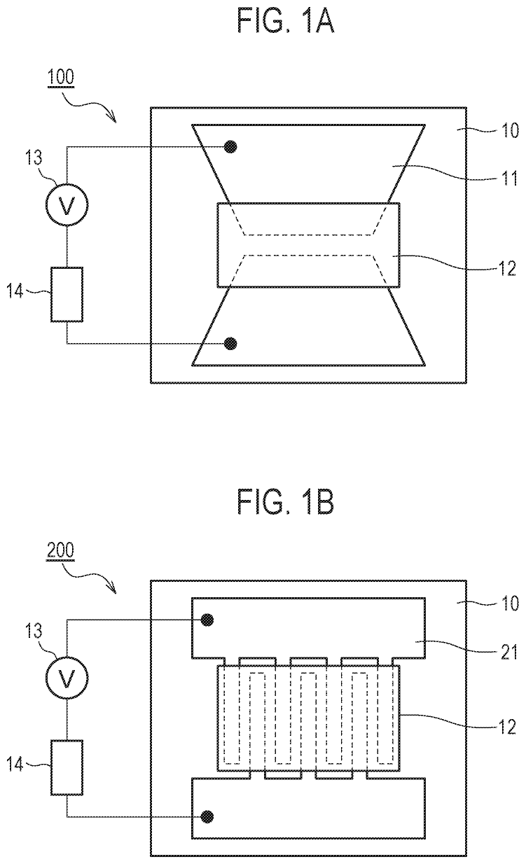

In this embodiment, a reducing gas sensor 100 (hereafter, simply referred to as “sensor 100”) configured to detect a reducing gas using a palladium metal complex (hereafter, referred to as “Pd complex”) will be described with reference to FIG. 1A. FIG. 1A is a schematic view illustrating the configuration of the sensor 100.

FIG. 1A is a schematic top view of the sensor 100 according to this embodiment. The sensor 100 includes a substrate 10, a pair of electrodes 11, a reactive layer (gas reaction section) 12, a power supply 13, and a measuring unit (detection circuit) 14.

The material for the substrate 10 may be, for example, glass, quartz, or silicon.

The pair of electrodes 11 are disposed on the substrate surface of the substrate 10 so as to oppose each other. The material for the pair of electrodes 11 is a conductor such as a metal or an organic conductive material. Specific examples include gold (Au), aluminum (Al), and ITO.

The pair of electrodes 11 can be appropriately designed so as to have a shape in accordance with, for example, the species of the reducing gas to be detected or the required sensitivity. For example, in the case of the configuration in FIG. 1A, the opposing parts of the pair of opposing electrodes 11 are linear. However, the shapes of the electrodes 11 are not limited to these, and may be, for example, comb shapes as illustrated, in FIG. 1B, in electrodes 21 of a sensor 200. The comb-shaped electrodes such as the pair of electrodes 21 can be designed to have a larger effective opposing electrode length of the pair of electrodes (electrode length) than linear electrodes, which enables measurement of the current of even materials having low electric conductivity.

The electrodes 11 and the electrodes 21 preferably have an electrode pitch of 0.05 μm or more and 100 μm or less, more preferably 0.05 μm or more and 30 μm or less, still more preferably 0.1 μm or more and 10 μm or less. Incidentally, the electrode pitch used herein is defined as the minimum distance of the distances between the pair of electrodes, in the opposing parts of the electrodes.

The reactive layer (gas reaction section) 12 is a layer disposed on the substrate surface of the substrate 10. The reactive layer (gas reaction section) 12 is disposed between the pair of electrodes 11 so as to be in electrical contact with each of the pair of electrodes 11. This electrical contact is not necessarily a physical contact: for example, such an electrode 11 and the gas reaction section 12 may be separated from each other or may have therebetween, for example, a conductive layer or a film permeable to the reducing gas as long as electrical signals (current) pass therebetween.

The gas reaction section 12 is a layer including a Pd complex. The Pd complex of the gas reaction section 12 is reduced by an irreversible redox reaction with the reducing gas. The gas reaction section 12 can be formed on the substrate surface of the substrate 10, from a solution in which the Pd complex is dissolved or suspended, by a method such as spin coating, dipping, casting, or bar coating. Alternatively, the gas reaction section 12 may be formed by vapor deposition under heating, for example. The Pd complex included in the gas reaction section 12 will be described later.

In the configurations illustrated in FIG. 1A and FIG. 1B, the gas reaction section 12 preferably has a film thickness of 5 nm or more and 1000 nm or less, more preferably 10 nm or more and 500 nm or less. Incidentally, in this embodiment, the gas reaction section 12 is formed as a monolayer, which is not intended to be limiting; the gas reaction section 12 may be formed as multilayers.

The gas reaction section 12 is disposed so as to come into contact with a gas including a reducing gas to be detected. When the gas reaction section 12 comes into contact with the gas including the reducing gas, the Pd complex included in the gas reaction section 12 undergoes an irreversible redox reaction with the reducing gas. This irreversible redox reaction causes a change in the electric conductivity of the gas reaction section 12. This change is measured with the measuring unit 14 connected to the electrodes 11.

The measuring unit 14 is a measuring unit that measures changes in the electric conductivity between the pair of electrodes 11. This measuring unit 14 is at least configured to measure changes in the electric conductivity of the gas reaction section 12 due to a redox reaction between the Pd complex of the gas reaction section 12 and the reducing gas. Specifically, the measuring unit 14 measures changes in at least one of the current between the pair of electrodes 11, the resistance between the pair of electrodes 11, the capacitance between the pair of electrodes 11, and the conductance between the pair of electrodes 11. The measuring unit 14 is electrically connected to each of the pair of electrodes 11, and uses, for example, an ammeter or a voltmeter to measure changes in the electric conductivity. The power supply 13 supplies voltage to the pair of electrodes 11.

The Pd complex used in the gas reaction section 12 according to this embodiment will be described. The Pd complex used in the gas reaction section 12 according to this embodiment is a Pd complex having a partial structure in which organic ligands including oxygen atoms and carbon atoms are coordinated to a divalent palladium atom (Pd(II)) serving as the central metal. In this case, four oxygen atoms are coordinated to the palladium atom serving as the central metal. Specifically, the material used in the gas reaction section according to this embodiment is a Pd complex including a partial structure represented by the following General formula (1).

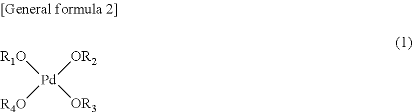

In General formula (1), each of OR₁ to OR₄ is a monodentate ligand, or forms a polydentate ligand by bonding tpo each other, or forms a bridging ligand by bonding to a palladium atom different from the palladium atom in General formula (1). R₁ to R₄ each have one or more carbons, and may be the same or different.

Incidentally, in this Description, “having a partial structure represented by a general formula” means that the Pd complex includes, at least in a portion of the Pd complex, the structure represented by the general formula. In other words, “having a partial structure represented by a general formula” means that the general formula represents the whole structure of the Pd complex, or the general formula represents a portion of the structure of the Pd complex.

Examples of the monodentate ligands represented by OR₁ to OR₄ include optionally substituted alkoxy groups, optionally substituted carboxylic acids, optionally substituted ketones, optionally substituted diketones, optionally substituted carboxylic acid amides, optionally substituted sulfoxides, and ionized forms of the foregoing.

Examples of the polydentate ligand formed by bonding among R₁ to R₄ of OR₁ to OR₄ include bidentate ligands in which R₁ and R₂ are bonded together and R₃ and R₄ are bonded together. Specific examples of the polydentate ligand formed by bonding among R₁ to R₄ of OR₁ to OR₄ include optionally substituted carboxylic acids, optionally substituted diketones, and ionized forms of the foregoing.

In the bridging ligands represented by OR₁ to OR₄, for example, R₁ and R₂, and R₃ and R₄ bridge the palladium atom in General formula (1) and palladium atoms different from the palladium atom. Specific examples of the bridging ligands represented by OR₁ to OR₄ include optionally substituted carboxylic acids, optionally substituted diketones, and ionized forms of the foregoing.

The Pd complex represented by General formula (1) according to this embodiment is a complex that can have various molecular structures. For example, the Pd complex represented by General formula (1) according to this embodiment can have a polynuclear complex structure represented by General formula (2).

[PdL₁L₂]ₙ (2)

In General formula (2), L₁ and L₂ are bridging ligands in which R₁ to R₄ in General formula (1) bond the palladium atom serving as the central metal in General formula (1) to palladium atoms different from the palladium atom, with oxygen atoms serving as coordinating atoms. L₁ and L₂ may be the same or different. Incidentally, n is an integer of 2 or more.

Each of L₁ and L₂ is independently selected from, for example, optionally substituted carboxylic acids, optionally substituted diketones, and ionized forms of the foregoing.

The Pd complex represented by General formula (2) has a polymeric structure. Examples of the polymeric structure include various structures such as, in the case of n=2, a dimer [PdL₁L₂]₂; in the case of n=3, a trimer [PdL₁L₂]₃; and in the case of n being 4 or more, a polymer [PdL₁L₂]ₙ. When the Pd complex is used for the sensor 100 according to this embodiment, a Pd complex having a polymeric structure may be used alone, or a mixture of different Pd complexes having polymeric structures may be used. Alternatively, a Pd complex having a monomeric structure may be used alone, or a mixture of one or more Pd complexes having polymeric structures and a Pd complex having a monomeric structure may be used.

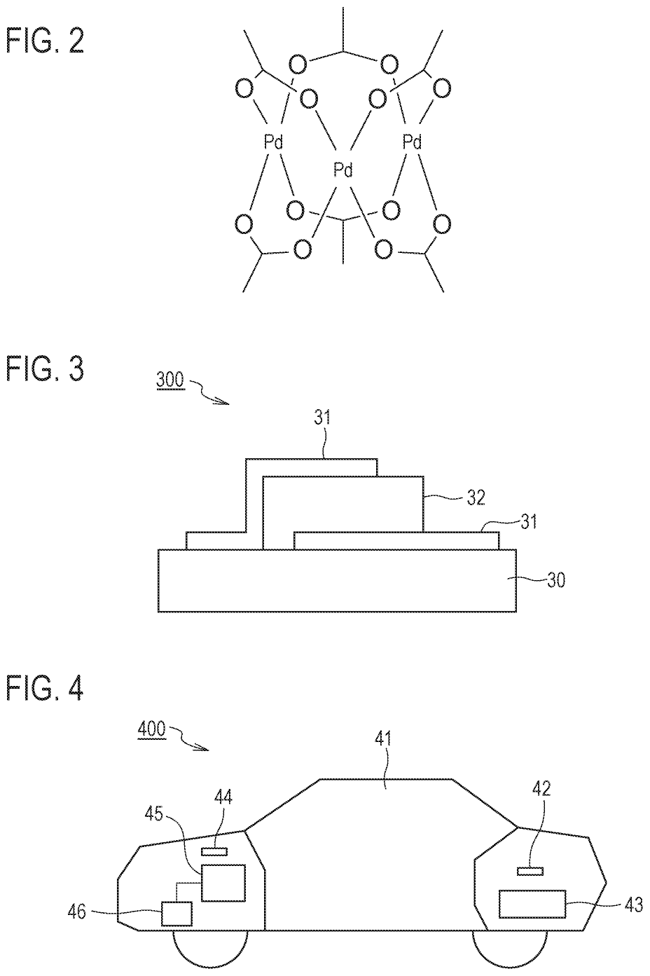

An example of the molecular structure of the Pd complex represented by General formula (2) is illustrated in FIG. 2 (Chem. Eur. J. 2016, DOI: 10.1002/chem.201601450). The Pd complex illustrated in FIG. 2 is a Pd complex in which bridging ligands L₁ and L₂ are ionized forms of acetic acid (CH₃COO⁻). Thus, the Pd complex represented by General formula (2) has a polynuclear structure in which the bridging ligands bridge a plurality of palladium atoms.

The bridging ligands L₁ and L₂ are preferably, for example, a carboxylate represented by the following General formula (3) and having the ionized form of a carboxylic acid.

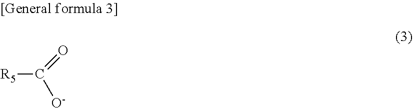

In General formula (3), R₅ represents an optionally substituted alkyl group, an optionally substituted aryl group, or an optionally substituted aralkyl group.

The optionally substituted alkyl group represented by R₅ may be linear, branched, or cyclic. Specific examples of the optionally substituted alkyl group represented by R₅ include a methyl group, an ethyl group, a normal propyl group, an isopropyl group, a normal butyl group, a tertiary butyl group, an octyl group, a cyclohexyl group, and a trifluoromethyl group.

Specific examples of the optionally substituted aryl group represented by R₅ include a phenyl group, a biphenyl group, a terphenyl group, a fluorenyl group, a naphthyl group, a fluoranthenyl group, an anthryl group, a phenanthryl group, a pyrenyl group, a tetracenyl group, a pentacenyl group, a triphenylenyl group, and a perylenyl group.

Specific examples of the optionally substituted aralkyl group represented by R₅ include a benzyl group and a phenethyl group. Incidentally, when the alkyl group, the aryl group, or the aralkyl group represented by R₅ has a substituent, it may have, as the substituent, at least one of a halogen atom, an alkyl group, or an alkoxy group.

The following are specific examples of structural formulas of the bridging ligands L₁ and L₂ represented by General formula (3). However, the bridging ligands L₁ and L₂ according to this embodiment are not limited to these.

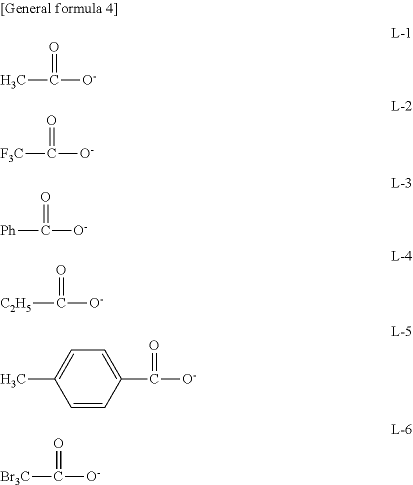

Table 1 describes example compounds having ligands L-1 to L-6 and being usable in the present invention. The indicative formulas are described in accordance with the descriptions provided by Aldrich or Tokyo Chemical Industry.

TABLE 1

| Compound No. | Ligand | Indicative formula |
|---|---|---|
| 1 | L-1 | Pd(L-1)₂ |
| 2 | L-1 | [Pd(L-1)₂]₃ |
| 3 | L-2 | Pd(L-2)₂ |
| 4 | L-3 | Pd(L-3)₂ |
| 5 | L-4 | Pd(L-4)₂ |
| 6 | L-5 | Pd(L-5)₂ |
| 7 | L-6 | Pd(L-6)₂ |

The Pd complex represented by General formula (1) according to this embodiment can have the structure of a chelate complex represented by General formula (4).

L₃-Pd-L₄ (4)

In General formula (4), each of L₃ and L₄ is a bidentate ligand having, as coordinating atoms, two oxygen atoms. L₃ and L₄ may be the same or different.

Specifically, the bidentate ligands represented by L₃ and L₄ are each independently selected from optionally substituted carboxylic acids, optionally substituted diketones, and ionized forms of the foregoing. L₃ and L₄ are each preferably a bidentate ligand represented by the following General formula (5). The bidentate ligand represented by the following General formula (5) is a diketonate ion provided by ionization of diketone.

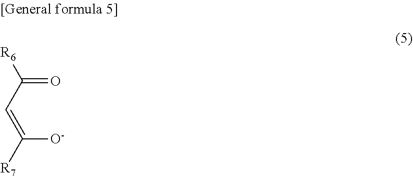

In General formula (5), R₆ and R₇ are each independently selected from optionally substituted alkyl groups, optionally substituted aryl groups, and optionally substituted aralkyl groups.

The optionally substituted alkyl groups represented by R₆ and R₇ may be linear, branched, or cyclic. Specific examples of the optionally substituted alkyl groups represented by R₆ and R₇ include a methyl group, an ethyl group, a normal propyl group, an isopropyl group, a normal butyl group, a tertiary butyl group, an octyl group, a cyclohexyl group, and a trifluoromethyl group.

Specific examples of the optionally substituted aryl groups represented by R₆ and R₇ include a phenyl group, a biphenyl group, a terphenyl group, a fluorenyl group, a naphthyl group, a fluoranthenyl group, an anthryl group, a phenanthryl group, a pyrenyl group, a tetracenyl group, a pentacenyl group, a triphenylenyl group, and a perylenyl group.

Specific examples of the optionally substituted aralkyl groups represented by R₆ and R₇ include a benzyl group and a phenethyl group. Incidentally, when the alkyl groups, the aryl groups, or the aralkyl groups represented by R₆ and R₇ have a substituent, they may have, as the substituent, at least one of a halogen atom, an alkyl group, or an alkoxy group.

The following are specific examples of the structural formulas of bidentate ligands represented by L₃ and L₄ in the Pd complex represented by General formula (4). However, the bidentate ligands represented by L₃ and L₄ according to this embodiment are not limited to these.

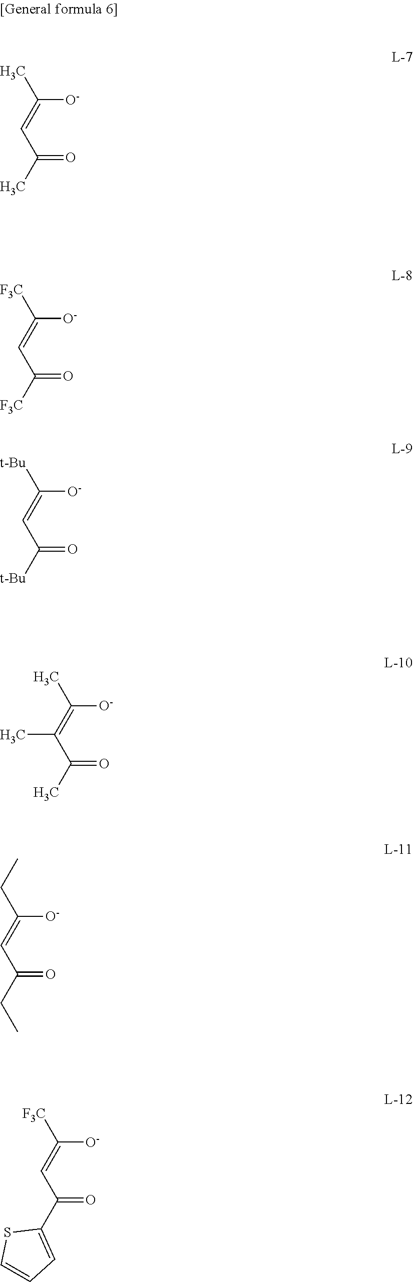

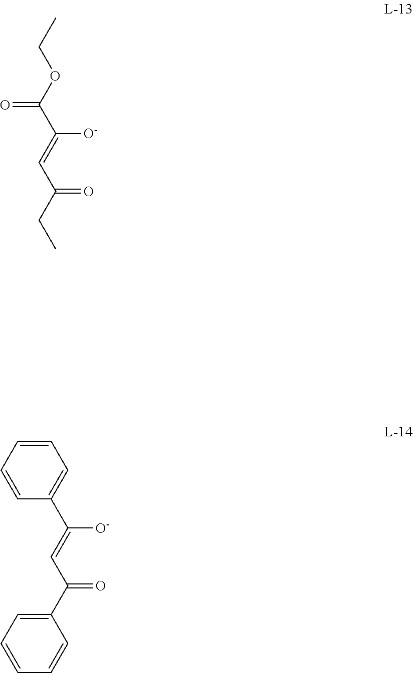

Table 2 describes example compounds having ligands L-7 to L-14 and being usable for the sensor 100.

TABLE 2

| Compound No. | Ligand | Pd complex |
|---|---|---|
| 8 | L-7 | Pd(L-7)₂ |
| 9 | L-8 | Pd(L-8)₂ |
| 10 | L-9 | Pd(L-9)₂ |
| 11 | L-10 | Pd(L-10)₂ |
| 12 | L-11 | Pd(L-11)₂ |
| 13 | L-12 | Pd(L-12)₂ |
| 14 | L-13 | Pd(L-13)₂ |
| 15 | L-14 | Pd(L-14)₂ |

When the Pd complex according to this embodiment, in which four oxygen atoms are coordinated to the palladium atom, comes into contact with a reducing gas, a reaction represented by Chemical reaction formula (a) or (b) occurs. Formula (a) describes an example of a reaction between the Pd complex according to this embodiment and hydrogen. Formula (b) describes an example of a reaction with ethylene.

Specifically, as described in Chemical reaction formula (a) or (b), the Pd complex according to this embodiment reacts with a reducing gas, so that the divalent palladium atom (Pd(II)) is reduced into a zerovalent palladium atom (Pd(0)).

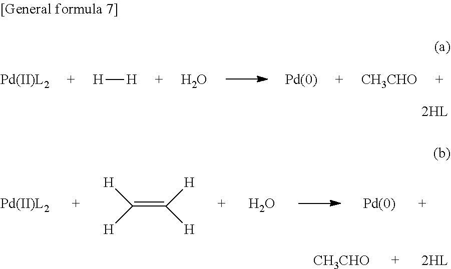

As described above, the Pd complex according to this embodiment reacts with a reducing gas to cause a change in the valence of the Pd atom serving as the central metal, from divalent palladium (Pd(II)) to zerovalent metallic palladium (Pd(0)). This reaction causes a considerable change in the electric conductivity. Specifically, for example, when a Pd complex film is formed in contact with a pair of electrodes and brought into contact with a reducing gas, a change in the electric conductivity is observed before and after the contact. This change in the electric conductivity can be used for detection of the reducing gas.

In the Pd complex represented by General formula (1), the palladium atom serving as the central metal has eight d electrons, and often takes a planar four-coordinated structure. Alternatively, as illustrated in FIG. 2, even under steric limitations, palladium atoms serving as central metals have a four-coordinated structure that is nearly planar. Thus, such a palladium atom is easily accessible to molecules of the reducing gas, which inferentially facilitates a redox reaction between the palladium atom and a molecule of the reducing gas, compared with the related art. Thus, use of the Pd complex represented by General formula (1) enables detection of the reducing gas at a high sensitivity.

As has been described so far, the sensor 100 measures a change in the electric conductivity of the gas reaction section 12 due to the reducing gas. Specifically, as described in Chemical reaction formulas (a) and (b) above, the valence of the palladium atom changes from Pd(II) (divalence) to Pd(0) (zerovalence). This change generates, from Pd(II) having a low electric conductivity, metallic Pd(0), which inferentially results in a very large change in the electric conductivity. In this embodiment, the Pd complex represented by General formula (1) is not used as a catalyst, but the Pd complex is used in the gas reaction section 12. A change in the electric conductivity of the reactive layer 12 due to an irreversible redox reaction between the Pd complex and the reducing gas is measured, to thereby detect the reducing gas. As a result, as described later in EXAMPLES, the change in the electric conductivity of the gas reaction section 12 results in a change in conductance of 10⁷ or more before and after the reaction with the reducing gas. This is a higher sensitivity than in the related art. The sensor 100, which uses the Pd complex represented by General formula (1) for the reactive layer, achieves a higher sensitivity than in sensors for detecting reducing gases in the related art.

In addition, the sensor 100 operates at a very low current before reacting with the reducing gas, which enables low power consumption during the normal operation. Thus, the sensor 100 achieves a reduction in the power consumption, compared with the related art. In addition, such low power consumption provides operation using a simple power supply such as a battery, which enables various applications.

Some hydrogen gas sensors in the related art require heaters during use. By contrast, the sensor 100 according to this embodiment enables detection of a reducing gas at ordinary temperature. Since heaters are not required, the configuration is simple, which contributes to a reduction in the size and a reduction in the costs.

Examples of the reducing gas in this embodiment include hydrogen, formaldehyde, carbon monoxide, ethylene, hydrogen sulfide, sulfur dioxide, and nitrous oxide. These are industrially useful, but most of them affect the human body when inhaled and hence need to be under safety control. Hydrogen is starting to be used as fuel for fuel cell cars and household fuel cells, and is considered as a promising energy source.

### Second Embodiment

In this embodiment, the configuration of a sensor 300 that detects a reducing gas will be described with reference to FIG. 3. FIG. 3 is a schematic sectional view illustrating the configuration of the sensor 300 according to this embodiment. The sensor 300 includes a substrate 30, a pair of electrodes 31, and a gas reaction section 32. The substrate 30 may be the same as the substrate 10 in the first embodiment.

The gas reaction section 32 is a layer disposed on the substrate surface of the substrate 30. The gas reaction section 32 is disposed so as to be sandwiched between the pair of electrodes 31 in a direction perpendicular to the substrate surface. Also in this embodiment, the gas reaction section 32 is a reactive layer including the Pd complex represented by General formula (1). Thus, as in the first embodiment, a power supply and a measuring unit (not shown) are connected to the pair of electrodes 31, the electric conductivity of the gas reaction section 32 is measured, and changes in the electric conductivity are monitored, to thereby detect the reducing gas. Incidentally, the gas reaction section 32 according to this embodiment preferably has a layer thickness of 5 nm or more and 1000 nm or less, more preferably 10 nm or more and 500 nm or less.

The sensor 300 according to this embodiment achieves a higher sensitivity than in sensors for detecting reducing gases in the related art. In addition, compared with the related art, a reduction in the power consumption is achieved, which provides operation with a simple power supply such as a battery. This enables various applications.

The sensor 300 according to this embodiment enables detection of the reducing gas at ordinary temperature. Since heaters are not required, the configuration is simple, which contributes to a reduction in the size and a reduction in the costs.

### Third Embodiment

In this embodiment, a moving body including the sensor 100, 200, or 300 according to the first embodiment or the second embodiment will be described. In this embodiment, a fuel cell car 400 serving as the moving body will be described with reference to FIG. 4. FIG. 4 is a schematic view illustrating an example of the configuration of the fuel cell car 400.

The fuel cell car 400 includes a car compartment 41, hydrogen gas sensors 42 and 44, a hydrogen fuel tank (hydrogen gas tank) 43, a fuel cell 45, and a motor 46.

The hydrogen fuel tank 43 and the fuel cell 45 are each disposed in a space sectioned off from the car compartment 41. The fuel cell 45 generates electric power upon supply of oxygen gas and hydrogen gas from the hydrogen fuel tank 43. The electric power generated by the fuel cell 45 is transmitted to the motor 46 of the fuel cell car 400, and used as the driving force for driving the fuel cell car 400. As the configuration of the fuel cell car 400, the generally known configuration of a fuel cell car can be employed.

In order to detect hydrogen gas as a reducing gas, the hydrogen gas sensors 42 and 44 are disposed close to the hydrogen fuel tank 43 and the fuel cell 45 and in the same spaces as those including the hydrogen fuel tank 43 and the fuel cell 45. The hydrogen gas sensors 42 and 44 detect (escaping) hydrogen gas outside of the flow path of hydrogen gas from the hydrogen fuel tank 43 to the fuel cell 45. Thus, the hydrogen gas sensors 42 and 44 detect hydrogen gas having leaked from the flow path of hydrogen gas including the hydrogen fuel tank 43 and the fuel cell 45. As the hydrogen gas sensors 42 and 44, the sensor 100 or 200 according to the first embodiment or the sensor 300 according to the second embodiment can be employed.

The hydrogen gas sensors 42 and 44 have higher sensitivity than in the related art and perform detection at low power consumption. Thus, irrespective of the usage state of the ignition key in the fuel cell car 400, leakage and the like of hydrogen gas can be always detected. Thus, hydrogen gas, which has been detected only in the turn-on state of the ignition key in the related art, can now be detected also in parked cars, for example. This enables safety control of fuel cell cars with more certainty.

Use of the above-described sensors according to the embodiments enables detection of the reducing gas at ordinary temperature. Since heaters are not required, the configuration is simple, which contributes to a reduction in the size and a reduction in the costs.

Incidentally, in this embodiment, as an example of the moving body, the fuel cell car is described. However, the moving body is not limited to this, and may be, for example, a motorcycle or drone including a fuel cell.

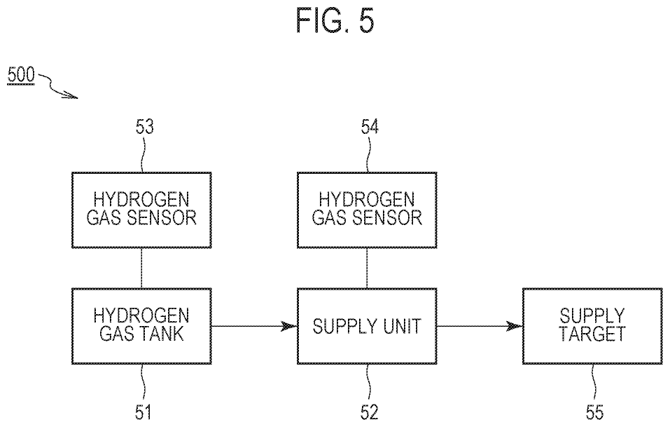

Alternatively, instead of the moving body, a hydrogen gas station, which stores hydrogen gas and supplies hydrogen gas to a supply target such as the hydrogen fuel tank of a moving body including a fuel cell, may include the sensor 100, 200, or 300 described in the first embodiment or the second embodiment. FIG. 5 is a block diagram illustrating the configuration of the hydrogen gas station. A hydrogen gas station 500 according to this embodiment includes a hydrogen gas tank 51, which stores hydrogen gas, a dispenser (supply unit) 52, which supplies hydrogen gas in the hydrogen gas tank 51 to a supply target 55, and hydrogen gas sensors 53 and 54, which detect hydrogen gas. The hydrogen gas sensors 53 and 54 detect (escaping) hydrogen gas having leaked from the flow path where hydrogen gas is supplied from the hydrogen gas tank 51 through the dispenser 52 to the supply target 55. As each of the hydrogen gas sensors 53 and 54, any one of the above-described sensors 100, 200, and 300 according to the embodiments may be employed.

### EXAMPLES

Hereinafter, Examples will be described. However, the present invention is not limited to the following Examples. In each of Examples, a sensor having the configuration of the sensor 200 according to the first embodiment was produced.

The sensors of Examples were evaluated. As the evaluation processes, a current response experiment and measurement of sensitivity S were performed.

In the current response experiment, while a voltage of 0.1 V was applied to the pair of electrodes and a gas mixture including a reducing gas to be detected was introduced near the sensor, changes in the current were measured. Changes in the current after introduction of the gas mixture were observed, and the time elapsed until a sharp change in the current started (response time) was determined. As the gas mixture, a gas mixture of 1% reducing gas/99% argon was employed. For example, when the detection-target reducing gas was hydrogen gas, a gas mixture of 1% hydrogen/99% air was used.

The sensitivity S was measured by measuring the conductance between the electrodes before the current response experiment, and the conductance between the electrodes after the current response experiment. From the measured conductances before and after the current response experiment, as in Nanotechnology, 21, 165503 (5 pp), 2010, the following Formula (2) was used to calculate sensitivity S. Regarding the sensors of Examples, current response time and sensitivity S are summarized in Table 3.

S=(G_{H}−G_{N})/G_{N} (2)

(G_{H}: conductance after reaction with reducing gas, G_{N}: conductance before reaction with reducing gas)

TABLE 3

| | | | | Electrode | Response | |
|---|---|---|---|---|---|---|
| | | | Production | pitch | time | Sensitivity |
| Example | Pd complex | Solvent | process | (μm) | (sec) | S |
| 1 | Pd(CH₃COO)₂ | Ethyl acetate | Spin coating | 5 | 280 | 10⁸ |
| 2 | | Ethyl acetate | Casting | 5 | 260 | 10⁸ |
| 3 | | Chloroform | Spin coating | 5 | 210 | 10⁸ |
| 4 | | Chloroform | Casting | 5 | 200 | 10⁸ |
| 5 | Pd₃(CH₃COO)₆ | Ethyl acetate | Spin coating | 10 | 280 | 10⁹ |
| 6 | | Ethyl acetate | Spin coating | 5 | 210 | 10⁹ |
| 7 | | Ethyl acetate | Spin coating | 1 | 110 | 10⁹ |
| 8 | | Ethyl acetate | Casting | 5 | 250 | 10⁸ |
| 9 | | Chloroform | Casting | 5 | 220 | 10⁸ |
| 10 | Pd(C₂H₅COO)₂ | Ethyl acetate | Spin coating | 5 | 310 | 10⁹ |
| 11 | | Ethyl acetate | Casting | 5 | 290 | 10⁹ |
| 12 | | Ethyl acetate | Casting | 3 | 220 | 10⁸ |
| 13 | | Ethyl acetate | Casting | 1 | 190 | 10⁸ |
| 14 | Pd(CF₃COO)₂ | Chloroform | Spin coating | 5 | 260 | 10⁷ |
| 15 | | Chloroform | Casting | 5 | 180 | 10⁸ |

### Example 1

In this Example, the sensor 200 according to the first embodiment was produced. As the Pd complex included in the gas reaction section 12, palladium acetate manufactured by Aldrich was used. A 1 wt % ethyl acetate solution of palladium acetate was prepared. The ethyl acetate solution of palladium acetate was applied by spin coating onto the electrodes 21 having comb shapes. The spin-coating conditions were 1000 revolutions/min for 30 seconds. The electrode pitch between the electrodes 21 was set to 5 μm. The electrode length of the electrodes 21 was set to 50 cm.

In the current response experiment, after 280 seconds elapsed from the introduction of a 1% hydrogen gas mixture, a sharp change in the current started; and then 10 seconds later, the current became constant. The sensitivity S was found to be 10⁸.

### Example 2

In this Example, the same method as in Example 1 was performed except that the gas reaction section was produced by a casting process, to produce the sensor 200. Specifically, on the electrodes 21, a 1 wt % ethyl acetate solution of palladium acetate as in Example was dropped, and dried for 10 minutes at room temperature to form the gas reaction section 12.

In the current response experiment, after 260 seconds elapsed from the introduction of a 1% hydrogen gas mixture, a sharp change in the current started; and then 10 seconds later, the current became constant. The sensitivity S was found to be 10⁸.

### Example 3

In this Example, a sensor was produced as in Example 1 except that, as the solvent for palladium acetate, chloroform was employed.

In the current response experiment, after 210 seconds elapsed from the introduction of a 1% hydrogen gas mixture, a sharp change in the current started; and then 10 seconds later, the current became constant. The sensitivity S was found to be 10⁸.

### Example 4

In this Example, a sensor was produced as in Example 2 except that, as the solvent for palladium acetate, chloroform was employed.

In the current response experiment, after 200 seconds elapsed from the introduction of a 1% hydrogen gas mixture, a sharp change in the current started; and then 10 seconds later, the current became constant. The sensitivity S was found to be 10⁸.

### Example 5

In this Example, a sensor was produced as in Example 1 except that, as the Pd complex, palladium acetate trimer (manufactured by Aldrich) was employed, and the electrode pitch of the electrodes 21 was set to 10 μm.

In the current response experiment, after 280 seconds elapsed from the introduction of a 1% hydrogen gas mixture, a sharp change in the current started. The sensitivity S was found to be 10⁹.

### Example 6

In this Example, a sensor was produced as in Example 1 except that, as the Pd complex, palladium acetate trimer (manufactured by Aldrich) was employed.

In the current response experiment, after 210 seconds elapsed from the introduction of a 1% hydrogen gas mixture, a sharp change in the current started. The sensitivity S was found to be 10⁹.

### Example 7

In this Example, a sensor was produced as in Example 1 except that, as the Pd complex, palladium acetate trimer (manufactured by Aldrich) was employed, and the electrode pitch of the electrodes 21 was set to 1 μm.

In the current response experiment, after 110 seconds elapsed from the introduction of a 1% hydrogen gas mixture, a sharp change in the current started. The sensitivity S was found to be 10⁹.

### Example 8

In this Example, a sensor was produced as in Example 6 except that a casting process was employed.

In the current response experiment, after 250 seconds elapsed from the introduction of a 1% hydrogen gas mixture, a sharp change in the current started. The sensitivity S was found to be 10⁸.

### Example 9

In Example 9, a sensor was produced as in Example 6 except that, as the solvent, chloroform was employed and a casting process was employed.

In the current response experiment, after 220 seconds elapsed from the introduction of a 1% hydrogen gas mixture, a sharp change in the current started. The sensitivity S was found to be 10⁸.

### Example 10

In this Example, a sensor was produced as in Example 1 except that, as the Pd complex, palladium propionate (manufactured by Aldrich) was employed.

In the current response experiment, after 310 seconds elapsed from the introduction of a 1% hydrogen gas mixture, a sharp change in the current started. The sensitivity S was found to be 10⁹.

### Example 11

In this Example, a sensor was produced as in Example 10 except that a casting process was employed to form the gas reaction section.

In the current response experiment, after 290 seconds elapsed from the introduction of a 1% hydrogen gas mixture, a sharp change in the current started. The sensitivity S was found to be 10⁹.

### Example 12

In this Example, a sensor was produced as in Example 10 except that a casting process was employed to form the gas reaction section, and the electrode pitch of the electrodes 21 was set to 3 μm.

In the current response experiment, after 220 seconds elapsed from the introduction of a 1% hydrogen gas mixture, a sharp change in the current started. The sensitivity S was found to be 10⁸.

### Example 13

In this Example, a sensor was produced as in Example 10 except that a casting process was employed to form the gas reaction section, and the electrode pitch of the electrodes 21 was set to 1 μm.

In the current response experiment, after 190 seconds elapsed from the introduction of a 1% hydrogen gas mixture, a sharp change in the current started. The sensitivity S was found to be 10⁸.

### Example 14

In this Example, a sensor was produced as in Example 3 except that, as the Pd complex, palladium(II) trifluoroacetate (manufactured by Aldrich) was employed.

In the current response experiment, after 260 seconds elapsed from the introduction of a 1% hydrogen gas mixture, a sharp change in the current started. The sensitivity S was found to be 10⁷.

### Example 15

In this Example, a sensor was produced as in Example 4 except that, as the Pd complex, palladium(II) trifluoroacetate (manufactured by Aldrich) was employed.

In the current response experiment, after 180 seconds elapsed from the introduction of a 1% hydrogen gas mixture, a sharp change in the current started. The sensitivity S was found to be 10⁸.

### Example 16

In this Example, the sensor of Example 1 was used to detect, instead of the hydrogen gas, ethylene gas. Thus, the current response experiment and the measurement of sensitivity S were performed using a gas mixture of 1% ethylene gas/99% air.

As a result, after 400 seconds elapsed from the introduction of the 1% ethylene-gas gas mixture, a sharp change in the current started. The sensitivity S was found to be 10⁹.

### Example 17

In this Example, the sensor of Example 1 was used to detect, instead of the hydrogen gas, formaldehyde. Thus, the current response experiment and the measurement of sensitivity S were performed using a gas mixture of 1% formaldehyde gas/99% air. After 630 seconds elapsed from the introduction of the 1% formaldehyde gas mixture, a sharp change in the current started. The sensitivity S was found to be 10⁹.

### Comparative Example 1

In Comparative Example, a sensor was produced as in Example 1 except that the palladium acetate in Example 1 was replaced by a Pd complex represented by the following General formula (6). In the sensor of this Comparative Example, an increase in the current was not observed in spite of introduction of the 1% hydrogen gas mixture.

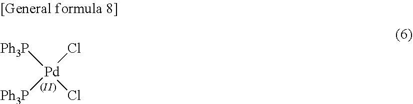

Examples 1 to 14 were each found to have a sensitivity S of 10⁷ or more, which is a higher sensitivity than in the related art. Thus, the sensors of Examples 1 to 14 each have a higher sensitivity than in the related art, and have a reduced power consumption. The sensors of Examples 1 to 14 each detected a reducing gas at room temperature. In other words, the sensors of Examples 1 to 14 do not require a heating unit such as a heater, which contributes to a reduction in the size of the device and simplification of the configuration.

In Examples 5 to 7 and Examples 11 to 13, with the changes in the electrode pitch, the response start time tended to change. Specifically, the smaller the electrode pitch, the shorter the response time.

A sensor according to an aspect of the present invention achieves, in a sensor configured to detect a reducing gas, a higher sensitivity than in the related art and a reduction in the power consumption.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A reducing gas sensor comprising:
a pair of electrodes; and
a reactive layer electrically in contact with the pair of electrodes,
wherein the reactive layer includes a palladium metal complex represented by General formula (1) below, and
the sensor is configured to measure a change in electric conductivity between the pair of electrodes, the change being caused by an irreversible redox reaction between the palladium metal complex and a reducing gas, to detect the reducing gas, 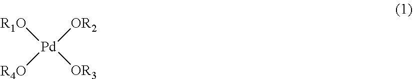
where each of OR₁ to OR₄ is a monodentate ligand, or forms a polydentate ligand by bonding to each other, or forms a bridging ligand by bonding to a palladium atom different from the palladium atom in General formula (1); and each of R₁ to R₄ has one or more carbons and may be the same or different.

2. The reducing gas sensor according to claim 1, further comprising:
a power supply configured to supply voltage to the pair of electrodes; and
a measuring unit configured to measure the change in electric conductivity between the pair of electrodes.

3. The reducing gas sensor according to claim 1, wherein the change in electric conductivity is measured by measurement of at least one of a current between the pair of electrodes, a resistance between the pair of electrodes, a conductance between the pair of electrodes, and a capacitance between the pair of electrodes.

4. The reducing gas sensor according to claim 1, wherein the palladium atom in General formula (1) is divalent palladium.

5. The reducing gas sensor according to claim 1, wherein each of OR₁ to OR₄ in General formula (1) is independently selected from optionally substituted alkoxy groups, optionally substituted carboxylic acids, optionally substituted ketones, optionally substituted diketones, optionally substituted sulfoxides, optionally substituted carboxylic acid amides, and ionized forms of the foregoing ones.

6. The reducing gas sensor according to claim 1, wherein the palladium metal complex is a polynuclear complex having a structure represented by General formula (2) below:
[PdL₁L₂]ₙ (2)
where each of L₁ and L₂ represents the bridging ligand, L₁ and L₂ may be the same or different, and n is an integer of 2 or more.

7. The reducing gas sensor according to claim 6, wherein each of L₁ and L₂ in General formula (2) above is a ligand represented by General formula (3) below: 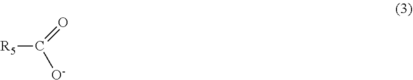
where R₅ represents an optionally substituted alkyl group, an optionally substituted aryl group, or an optionally substituted aralkyl group.

8. The reducing gas sensor according to claim 1, wherein the palladium metal complex is a chelate complex having a structure represented by General formula (4) below:
L₃-Pd-L₄ (4)
where each of L₃ and L₄ is a bidentate ligand in which two oxygen atoms are coordinated to the palladium atom, and L₃ and L₄ may be the same or different.

9. The reducing gas sensor according to claim 8, wherein each of L₃ and L₄ in General formula (4) is a bidentate ligand represented by General formula (5) below: 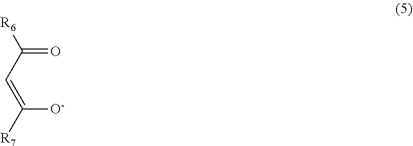
where each of R₆ and R₇ is independently selected from optionally substituted alkyl groups, optionally substituted aryl groups, and optionally substituted aralkyl groups.

10. The reducing gas sensor according to claim 1, wherein the reducing gas is hydrogen gas.

11. The reducing gas sensor according to claim 1, wherein each of the pair of electrodes has a comb shape.

12. A moving body comprising:
a hydrogen gas tank configured to store hydrogen gas;
a fuel cell configured to generate electric power upon supply of oxygen gas and the hydrogen gas from the hydrogen gas tank;
a motor configured to be driven by the electric power generated by the fuel cell; and
a hydrogen gas sensor configured to detect escaping hydrogen gas,
wherein the hydrogen gas sensor includes the reducing gas sensor according to claim 1.

13. A hydrogen gas station configured to supply hydrogen gas, the hydrogen gas station comprising:
a hydrogen gas tank configured to store hydrogen gas;
a supply unit configured to supply hydrogen gas from the hydrogen gas tank to a target; and
a hydrogen gas sensor configured to detect escaping hydrogen gas,
wherein the hydrogen gas sensor includes the reducing gas sensor according to claim 1.
